(19) United States
(12) Patent Application Publication
(10) Pub. No.: US 2006/0074294 A1
(43) Pub. Date: Apr. 6, 2006

(54) Medical imaging system, dispensing system, method, and computer program product for assessing patient renal function prior to dispensing a contrast media as part of a medical imaging procedure

(75) Inventors: Robert C. Williams JR., Fort Salonga, NY (US); Brad Schreck, Totowa, NJ (US); Jeff Cushner, Woodmore, NY (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: E-Z-EM, Inc.

(21) Appl. No.: 10/959,466

(22) Filed: Oct. 6, 2004

(51) Int. Cl. A61B 5/05

(52) U.S. Cl. 600/420

(57) ABSTRACT

A medical imaging system, dispensing system, method, and computer program product for analyzing biological fluid chemistry as part of a medical imaging procedure are provided. The system of the present invention provides a biological fluid analyzer configured to analyze a biological fluid sample for the presence of specific components that indicate patient's ability to safely process and clear injected contrast media from the vasculature that may be injected as part of the medical imaging procedure. The medical imaging system, dispensing system, and method for analyzing biological fluid chemistry are further provided as part of the medical imaging procedure and/or medical imaging suite so as to be capable of determining in real time, a patient's ability to safely clear injected contrast media.

### FIELD OF THE INVENTION

The present invention relates generally to the analysis of patient biological fluid chemistry prior to a medical imaging procedure that requires the injection of a contrast media. More specifically, the present invention relates to an analysis of biological fluid chemistry risk factors indicating a possible deficiency in renal function in a patient prior to the injection of a contrast media used in a medical imaging procedure. The present invention provides a system, method, and device that may be integrated into a medical imaging suite for analyzing biological fluid chemistry risk factors indicating a possible deficiency in renal function in a patient prior to the injection of a contrast media.

### BACKGROUND OF THE INVENTION

Medical imaging procedures often rely on the use of a contrast media that is injected into the biological structure to be imaged such that the medical imaging procedure provides more detailed information to a radiologist or other medical personnel responsible for analyzing the medical imagery. Contrast media is often injected into a patient's vasculature prior to the medical imaging procedure such that the patient's renal system is thereafter tasked with clearing the contrast media from the patient's bloodstream.

According to conventional radiographic diagnostic imaging techniques, such as X-ray procedures, X-rays pass through a target object and expose an underlying photographic film. The developed film then provides an image of the radiodensity pattern of the object. Less radiodense areas produce a greater blackening of the film; more radiodense, bony tissues produce a lighter image. Effective contrast media for X-ray may be either less radiodense than body tissues or more radiodense. The less radiodense agents include air and other gases; an example of a more radiodense contrast material is a barium sulfate suspension or iodinated injectable media.

Computed tomography (CT) is superior to conventional radiography in its ability to image, with extremely high resolution, a succession of thin sections of an object at specific points, lines or planes along the X, Y, or Z axis of the target object. However, because this procedure is also based on the detection of differences in radiodensity, requirements for contrast media in CT are essentially identical with those for conventional radiography.

Magnetic resonance imaging (MRI) systems for body imaging operate on a different physical principle. Generally, MRI relies on the atomic properties (nuclear resonance) of protons in tissues when they are scanned with radio frequency radiation. The protons in the tissue, which resonate at slightly different frequencies, produce a signal that a computer uses to tell one tissue from another. MRI provides detailed three-dimensional soft tissue images.

Fluoroscopy imaging systems may provide real-time X-ray images of internal structures based on differences in the radiodensity of the imaged object components. As in X-ray procedures, fluoroscopy may be enhanced by the use of more radiodense contrast media that may be injected into the object being imaged. For instance, in angiography procedures, radiodense contrast media may be injected into the cardiac vasculature in order to trace the path of blood through the vasculature and determine, for instance, the location of blockages in the cardiac vasculature.

Currently, injection systems used for the dispensing of a contrast media in, for instance, CT, MRI, Ultrasound and/or Angiography/Fluoroscopy medical imaging procedures include interface controls and features limited to the delivery of contrast media within the medical imaging suite. Further, most contrast media is injected to a patient's vasculature for enhancement of imaging procedures and is then physiologically cleared by the renal system through normal nephritic function. During the clearing of contrast media from the patient's body, the serum-borne contrast media places additional burden on renal function until it is cleared. In cases where a patient undergoing a medical imaging procedure using contrast media has a prior history or an unknown pre-existing condition of compromised or impaired renal function, the burden associated with clearing injected contrast media can result in further damage to the kidneys and/or other components of the renal system. Furthermore, in some severe cases, the burden associated with the clearing of iodinated contrast media has destroyed renal function in its totality.

It is possible, however, to perform a blood test whereby blood urea nitrogen (BUN) and creatinine levels can be measured as a method for assessing renal function and a patient's ability to safely clear contrast media. However, current medical imaging systems, such as contrast media injection equipment in existing medical imaging suites, do not provide for the clinical biological fluid chemistry measurements of BUN and creatinine to pre-screen and/or qualify a patient for contrast media injection. In addition, the measurements of BUN and creatinine levels are not made on a substantially real-time basis in the medical imaging suite as part of a medical imaging procedure.

For example, in current inpatient hospital settings, the clinical chemistry laboratory is typically located in a different area of the hospital from the radiology department. As such, either the patient, or a biological fluid sample from the patient must be forwarded to the clinical chemistry laboratory for processing. In the case where a biological fluid sample is transferred to the clinical laboratory, the additional phlebotomist time and expense is incurred. Thereafter, the results must be reported and either transmitted directly to the radiologist from the lab, or indirectly to the radiologist through the referring physician prescribing the radiographic exam in the first place. In short, the logistics of patient routing and transmission of the patient's laboratory results for BUN and creatinine is cumbersome. Similar obstacles are encountered for patients requiring pre-qualifying biological fluid BUN/creatinine analysis prior to undergoing contrast enhanced radiographic examination in an outpatient radiology practice. In this case, the clinical laboratory and radiology office may be in separate buildings separated by large geographic distances.

Thus, there exists a need for a medical imaging system, dispensing system, and method for determining, as part of a medical imaging procedure, the presence of biological fluid sample components to assess renal function in a patient scheduled for a medical imaging procedure. There further exists a need for a medical imaging system, dispensing system, and method that may be utilized within a medical imaging suite so that a prospective medical imaging patient may be pre-screened, preferably in real-time, for possible compromised and/or impaired renal function that may be exacerbated by the injection and subsequent clearing of contrast media dispensed to the patient prior to and/or during a medical imaging procedure.

### SUMMARY OF THE INVENTION

The above and other needs are met by the present invention which, in one embodiment, provides a medical imaging system comprising a medical imaging device configured to provide an image of a patient using a contrast media dispensed to the patient, a dispensing device configured to dispense the contrast media to the patient, and an analyzing device adapted to receive and analyze a biological fluid sample from the patient so as to determine a level of at least one substance in the biological fluid sample. The analyzing device may be further adapted to advise an operator of the system of the level of the at least one substance, and to advise the operator to dispense the contrast media if the level of the at least on substance is within a selected range. The at least one substance, may in some embodiments, comprise BUN, creatinine, and combinations thereof such that the systems and method of the present invention may aid in the assessment of a patient's renal function prior to the dispensing of a contrast media as part of a medical imaging procedure.

According to other advantageous embodiments the analyzing device may be further configured to communicate with the dispensing device so as to send the level of the at least one substance to the dispensing device. Furthermore, the dispensing device may be further configured to receive the level of the at least one substance and to dispense the contrast media to the patient if the level of the at least one substance is within the selected range. In some embodiments, the medical imaging device, the dispensing device, and the analyzing device may be co-located in a medical imaging suite so as to determine the level of the at least one substance in the medical imaging suite prior to a medical imaging procedure.

In additional embodiments, the analyzing device may further comprise a testing device configured to be in fluid communication with the biological fluid sample such that the testing device may provide a visual indicia to advise the operator of the system of the level of the at least one substance relative to the selected range. In another embodiment, the analyzing device may further comprise a testing device configured to receive the biological fluid sample and to be in fluid communication with the biological fluid sample, and a computer device configured to receive the testing device and to become operably engaged with the testing device to determine the level of the at least one substance in the biological fluid sample.

Some embodiments of the present invention may also provide a dispensing system adapted to dispense a contrast media used in a medical imaging procedure. The dispensing system may comprise, for instance, a dispensing device configured to dispense the contrast media to a patient, and an analyzing device adapted to receive and analyze a biological fluid sample from the patient so as to determine a level of at least one substance in the biological fluid sample. Furthermore, the analyzing device may be further adapted to advise an operator of the system of the level of the at least one substance and advise the operator to dispense the contrast media if the level of the at least on substance is within a selected range.

According to the method and computer program product embodiments of the present invention, a method for assessing the renal function of a patient prior to the dispensing of a contrast media as part of a medical imaging procedure is provided. The method comprises the steps of: collecting a biological fluid sample from the patient; determining a level of at least one substance in the biological fluid sample of the patient using an analyzing device located in a medical imaging suite; comparing the level of the at least one substance to a selected range of levels of the at least one substance using the analyzing device located in the medical imaging suite; and advising an operator of the analyzing device as to whether the level is within the selected range such that the operator may be advised of the patient's renal function prior to dispensing a contrast media without the need to send the patient and/or the biological fluid sample outside of the medical imaging suite for renal function testing.

According to other method embodiments, the method may further comprise the step of dispensing the contrast media to the patient if the level of the at least one substance is within the selected range such that the patient is screened for substantially normal renal function prior to dispensing the contrast media. According to other method embodiments, the determining step may further comprise determining a level of blood urea nitrogen (BUN), creatinine, or combinations thereof in the biological fluid sample.

Such embodiments provide significant advantages as described and otherwise discussed herein.

### BRIEF DESCRIPTION OF THE DRAWINGS

Having thus described the invention in general terms, reference will now be made to the accompanying drawings, which are not necessarily drawn to scale, and wherein:

FIG. 1 shows one embodiment of the medical imaging system of the present invention wherein the medical imaging device, dispensing device, and analyzer device are co-located within a medical imaging suite;

FIG. 2 shows one embodiment of the medical imaging system and dispensing system of the present invention wherein the analyzing device is in communication with the dispensing device;

FIG. 3 shows one embodiment of the medical imaging system of the present invention wherein the analyzing device is in communication with the dispensing device, medical imaging device, and/or a memory device via a network; and

FIG. 4 shows one embodiment of the medical imaging system and dispensing system of the present invention wherein the analyzing device comprises a self-contained consumable test strip.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

While the embodiments of the medical imaging system, dispensing system and method for assessing patient renal function prior to a medical imaging procedure are described below in the context of assessing renal function via the determination of a level of at least one substance in a biological fluid sample, it should be understood that the embodiments of the present invention may also be utilized to determine a level and/or the presence of, a variety of substances that may be present in a biological fluid sample so as to assess a patient's ability to safely ingest and/or receive an injection of a contrast media prior to undergoing a medical imaging procedure. The system and method embodiments of the present invention may be used for instance, to provide the capacity to determine a level and/or presence of a variety of substances in a biological sample within, for instance, a medical imaging facility, such that the determination may occur in substantially real time so as to minimize delays that may occur in pre-screening a prospective patient prior to a medical imaging procedure.

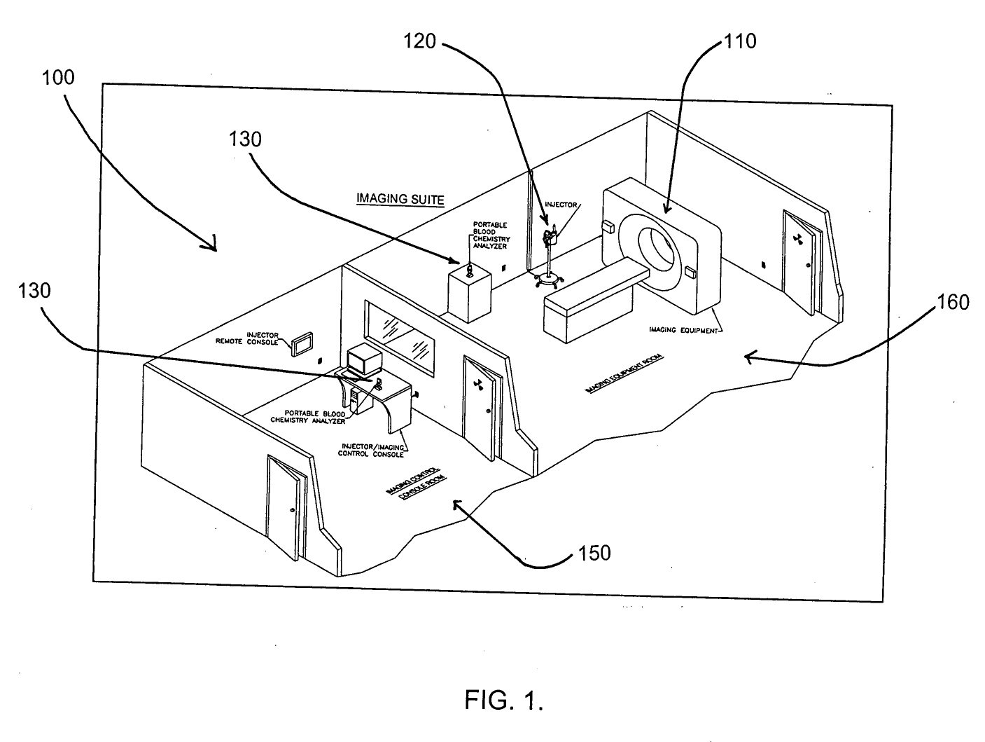

FIG. 1 shows a medical imaging system according to one embodiment of the present invention wherein a medical imaging device 110 is located within a medical imaging suite 100 of a hospital, health care facility, and/or research facility. The medical imaging device of the present invention may comprise, for instance, a computed tomography (CT) scanner, a fluoroscope, a positron emission tomography (PET) scanner, a magnetic resonance (MR) scanner, an ultrasound device and/or other imaging device that may require the dispensing of a contrast media to a patient prior to performing the medical imaging procedure so as to enhance the quality of an image produced by the imaging device 110. As used herein, the term “medical imaging suite” 100 refers generally to a room or collection of rooms within, for instance, a hospital or other health care facility, wherein various components of a medical imaging system may be located. The medical imaging suite 100 may further comprise, for instance, a control room 150 where an operator of the medical imaging system may be stationed, as well as an imaging room 160 wherein the medical imaging device 110 and other equipment related to a medical imaging procedure may be located. One skilled in the art will appreciate that the medical imaging device 110 may further comprise a computer device operably engaged with the medical imaging device so as to control the operation of the medical imaging device 110 via, for instance, a remotely located controller computer device, that may be located, for instance, in the control room 150 of the medical imaging suite. As such, the medical imaging device 110 may be controlled remotely by an operator of the medical imaging system and the medical imaging device may be further in communication with a computer network via wire connection and/or wireless methods such that images provided by the medical imaging device may be sent to the controller computer device such that the images may be adapted to be viewed by an operator of the medical imaging system and/or stored in a memory device operably engaged with the controller computer device in the control room 150. As described below, the medical imaging device 110 may further be configured to be in communication with other components of the medical imaging system of the present invention via, for instance, a computer network, such that data related to a given patient and/or medical imaging procedure may be transferred between the components of the medical imaging system of the present invention and/or to other electronic devices connected to or otherwise in communication with the computer network.

FIG. 1 also shows a dispensing device 120 located within the imaging room 160, for administering contrast media to a patient prior to being subjected to a medical imaging procedure. The dispensing device 120 may be configured to dispense a contrast media that is adapted to be ingested orally by the patient being subjected to the medical imaging procedure, such as, for instance, liquid iodine. The dispensing device 120 may, in some advantageous embodiments, be an injection device, such as, for instance a power injector, configured to inject a contrast media directly into the vasculature of the patient prior to the inception of the medical imaging procedure. In some embodiments, the dispensing device 120 may further comprise a computer device operably engaged therewith, wherein the computer device may be configured to be connected via wire connection or wireless methods to a computer network. Thus, the dispensing device 120 may be controlled remotely by an operator of the medical imaging system by, for instance, a controller computer device, configured to communicate via the computer network, with the dispensing device 120 such that he dispensing device 120 may be located in the imaging room 160 while the operator of the medical imaging system may control the dispensing device 120 from, for instance, a control room 150 adjacent to the imaging room 160 or located elsewhere within the medical imaging suite 100.

Also shown in FIG. 1 is an analyzing device 130, co-located with the medical imaging device 110, and the dispensing device 120, within the medical imaging suite 100. The analyzing device 130, according to embodiments of the present invention, may be adapted to receive and analyze a biological fluid sample from the patient so as to determine a level of at least one substance in the biological fluid sample prior to the dispensing of contrast media by, for instance, the dispensing device 120. The biological fluid sample may comprise, for instance, a blood sample, urine sample, saliva sample, and/or other biological fluid samples suitable for analysis in the analyzing device 130. In some advantageous embodiments, the analyzing device 130 may be further adapted to advise an operator of the system of the level of the at least one substance, and to advise the operator to dispense the contrast media (via, for instance, the dispensing device 120 or alternatively imaging device 110), if the level of the at least on substance is within a selected range and/or above or below a selected threshold level. As such, the analyzing device 130 may, in some advantageous embodiments, provide for a substantially real-time determination of the level of the at least one substance so as to allow the operator of the medical imaging system (and/or other medical personnel) to assess, for instance, the ability of the patient to safely be injected with the contrast media, prior to the inception of the medical imaging procedure. For instance, in some embodiments, the analyzing device 130 of the present invention may determine a level of blood urea nitrogen (BUN) and/or creatinine in a blood sample taken from a prospective patient so as to assess the ability of the prospective patient to safely clear the contrast media from their vascular system without causing damage to the renal system of the prospective patient. One skilled in the art will appreciate that determination of BUN and/or creatinine levels may allow medical personnel to assess the prospective patient's renal function and thereby preliminarily determine the prospective patient's ability to clear dispensed contrast media via the patient's renal system. The analyzing device may, however, be further configured to detect and/or determine a level of a variety of substances within a biological fluid sample taken from a prospective patient so as to assess the patient's suitability to be subjected to a particular type of medical imaging procedure without requiring the patient or a biological fluid sample associated with the patient to be sent outside of the medical imaging suite 100.

As shown in FIG. 1, the analyzing device 130 of the present invention may, in some embodiments, also be located in a control room 150 of the medical imaging suite 100 such that an operator of the medical imaging system may obtain a biological fluid sample from a prospective patient located, for instance in the imaging room 160 and subsequently bring the biological fluid sample into contact with the analyzing device 130 within the control room 150 so as to determine a level of at least one substance in the biological fluid sample prior to initiating the dispensing of contrast media. As described above, the analyzing device 130 may be further adapted to advise the operator to dispense the contrast media (via, for instance, the dispensing device 120 or alternatively imaging device 110), if the level of the at least one substance is within a selected range. The selected range may, for instance, be indicative of a range of levels of the at least one substance indicating that the patient has a substantially normal renal function which would allow the patient to safely clear the contrast media from their bloodstream. According to this embodiment, if the level of the at least one substance is within the selected range, the operator may then remotely initiate the medical imaging procedure from the control room 150, by for instance, remotely controlling the dispensing device 120 to dispense the contrast media to the patient and subsequently remotely controlling the medical imaging device 110 to provide an image of the patient. This embodiment may be suitable for minimizing unnecessary radiation exposure to the operator if, for instance, the medical imaging procedure utilizes radioactive emissions to provide an image, and/or in embodiments wherein the contrast media to be dispensed comprises a radioactive substance.

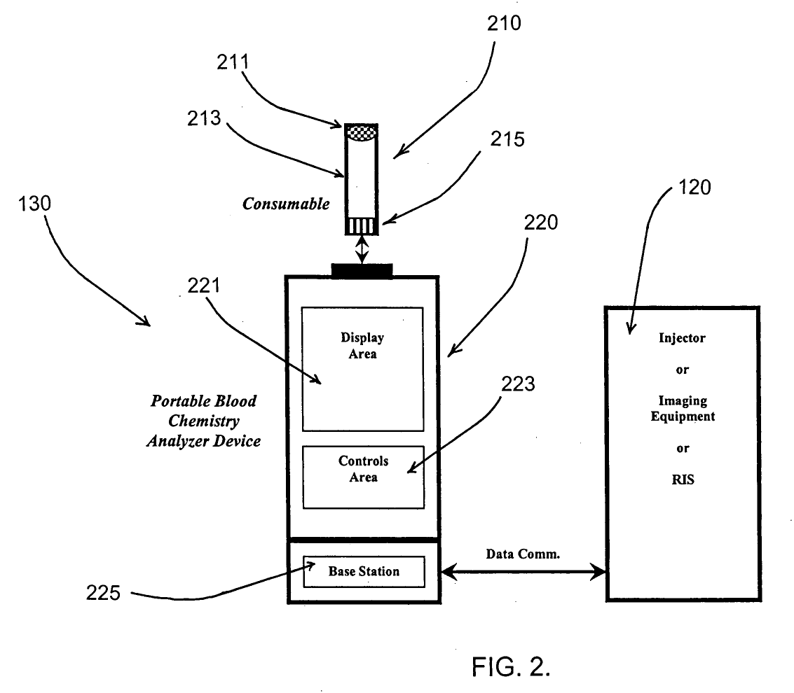

FIG. 2 shows a schematic representation of the analyzing device 130 according to one embodiment of the present invention. As shown, the analyzing device may further comprise a testing device 210 configured to receive and be in fluid communication with a biological fluid sample taken from a prospective patient and a computer device 220 configured to receive the testing device 210 and to become operably engaged therewith to determine the level of the at least one substance in the biological fluid sample. The testing device 210 may further comprise, for instance, a biological fluid sample collection reservoir 211, at least one reagent 213 configured to interact with the at least one substance, and a connecting (this may be a better choice of words from a technical standpoint) device 215 configured to communicate with the computer device 220 such that the computer device 220 may further determine the level of the at least one substance in the biological fluid sample. The biological fluid sample collection reservoir may further comprise, for instance, a plurality of capillaries configured to receive the biological fluid sample and transfer, via, for instance, capillary action, the biological sample to a portion of the biological fluid sample collection reservoir 211 containing at least one reagent 213 or other biochemical material suitable for reacting with the biological fluid sample for the purposes of determining a level of the at least one substance. For instance, in some embodiments, the reagent 213 may react with a biological fluid sample to produce a color change, and/or ionization, and/or electro-chemical reaction within the testing device 210 such that the connecting device 215 may transmit, for instance, the degree of color change, and/or ionization, and/or electro-chemical reaction occurring within the testing device to a computer device 220 (as described more fully below) via for instance, electrical, and/or electro-optical, and/or electro-chemical methods such that the computer device 220 may determine the level of the at least one substance in the biological fluid sample. According to some embodiments, the testing device 210 may comprise a consumable test strip further comprising the biological fluid sample collection reservoir 211, reagent 213, and connecting device 215 as described above such that each testing device 210 may be discarded after determining the level of the at least one substance in a given biological fluid sample. Thus, a new testing consumable test strip may be used for analyzing a biological fluid sample from each prospective patient entering the medical imaging suite 100.

Also, as shown in FIG. 2, the analyzing device 130 may further comprise a computer device 220, as described generally above, which may be re-used and configured to receive a testing device 210 corresponding to a biological fluid sample related to each prospective patient entering the medical imaging suite 100. The computer device 220 may be further configured to communicate with the testing device 210 via, for instance, the transmitting device 215 operably engaged with the testing device 210. As described above, the computer device may communicate with the testing device 210 via electrical, and/or electro-optical, and/or electrochemical methods so as to determine a degree of reaction of the reagent 213 with the biological fluid sample so as to determine a level of the at least one substance in the biological fluid sample. Also, as shown in FIG. 2, the computer device 220 may further comprise a display 221 and an input device 223. The computer device 220 may further comprise a memory device such that an operator of the medical imaging system may enter, via, for instance, the input device 223 data related to the medical imaging procedure, including patient information, the selected range of the level of the at least one substance, and/or other information related to the medical imaging procedure. Thus, the computer device 220 may compare the level of the at least one substance in the biological fluid sample as determined by the testing device 210 with the selected range entered by an operator of the medical imaging system, so as to advise the operator of the medical imaging system, via, for instance, the display 221, whether or not the operator may safely dispense the contrast media to the patient. The computer device 220 may comprise a variety of electronic devices including, for instance, a personal computer (including laptop personal computers), a PDA, palmtop computer devices, and/or other computer devices suitable for operable engagement with the base station 225 and/or the testing device 210. In addition, the display 221 may comprise, for instance, a cathode ray tube (CRT), LCD, LCD touch-screen, or other display device suitable for displaying text, images, graphics, and/or numerical data related to the medical imaging procedure and the level of the at least one substance in the biological fluid sample relative to the selected range input by, for instance, an operator of the medical imaging system.

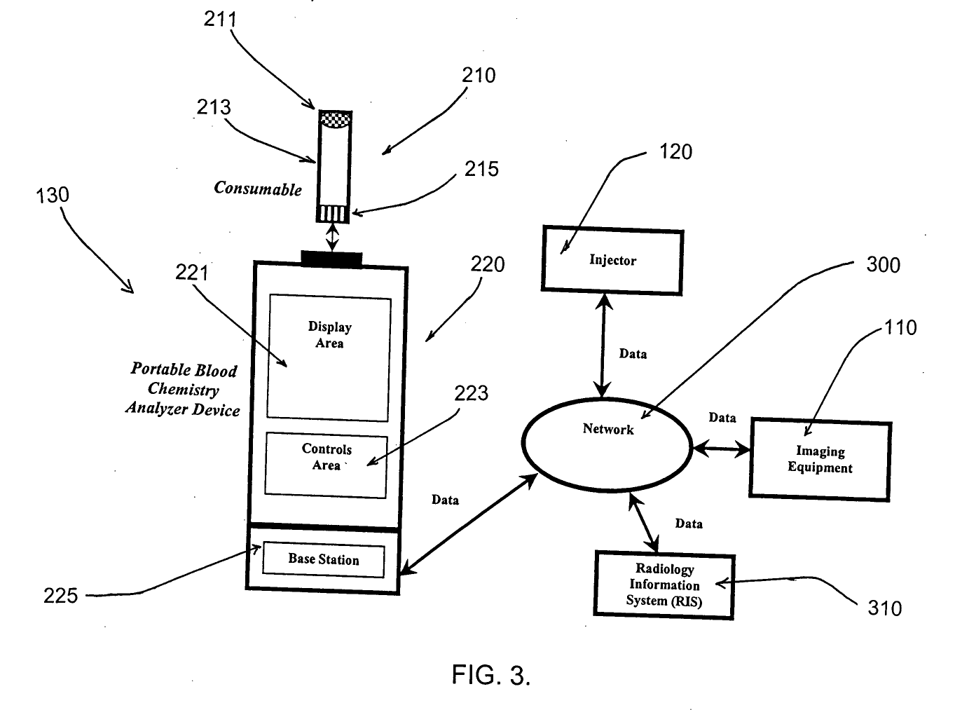

As shown in FIGS. 2 and 3, the computer device 220 may be configured to become operably engaged with a base station 225 such that the computer device 220 may be removed from the base station and carried by, for instance, an operator of the medical imaging system. Thus, in this embodiment, the computer device may be carried by an operator of the medical imaging system so as to allow the operator to obtain a biological fluid sample from the prospective patient and determine a level of the at least one substance in the biological fluid sample in substantially real time within the medical imaging suite 100. The operator may then return the computer device to an operable engagement with the base station 225. According to other advantageous embodiments, the base station may be a wireless network node, such that the computer device may remain in communication with the base station even as the computer device 220 is carried throughout the medical imaging suite 100. The base station 225 may also comprise various types of network devices, such as a network node and/or router, such that when the computer device 220 becomes operably engaged with the base station 225, the computer device 220 may be further configured to communicate with the computer network 300. As shown in FIG. 3, the computer device 220 may be also configured to be in communication with the medical imaging device 110 and/or the dispensing device 120 via, for instance, the computer network 300. Furthermore, according to some embodiments, the analyzing device 130 (and associated computer device 220) may be further configured to be in communication (via, for instance, the computer network 300) with the dispensing device 120 so as to be capable of sending the level of the at least one substance to the dispensing device 120. Furthermore, the dispensing device 120 may be configured to receive the level of the at least one substance and to dispense the contrast media to the patient if the level of the at least one substance is within the selected range. This embodiment may also provide for an operator lock-out feature such that if, for instance, the operator attempts to dispense a contrast media and/or initiate a medical imaging procedure wherein the determined level of the at least one substance is outside of the selected range (which may indicate that the prospective patient is not suited to receive the contrast media) the analyzing device 130 may send a lock-out signal to the dispensing device 120 such that the operator may not dispense the contrast media before entering an override code. The lock-out feature may be accomplished for instance, by sending an electronic signal, via the computer network 300 from the computer device 220 to the dispensing device 120 (or a computer device operably engaged therewith) that the level of the at least one substance in the biological fluid sample is outside the selected range corresponding to a patient's ability to safely clear a given contrast media. According to other embodiments of the present invention, an electronic signal may also be sent, via the computer network 300, from the computer device 220 to the medical imaging device 110 to lock-out an operator of the medical imaging system when the level of the at least one substance in the biological fluid sample is outside the selected range corresponding to a patient's ability to safely clear a given contrast media. These lock-out features may provide an additional safety feature to some embodiments in order to prevent the dispensing of contrast media to a prospective patient exhibiting levels of the at least one substance outside of the selected range, which may, in turn, indicate that the patient may have difficulties in safely clearing contrast media from their bloodstream via, for instance, the renal system.

In some embodiments, the medical imaging system of the present invention may further comprise a database 310 configured to store data related to individual patient histories, the level of the at least one substance in the biological fluid sample for past screenings of patients, as well as storing selected range data suitable for screening for substantially normal renal function and/or other physiological information pertinent to assessing a prospective patient's eligibility to receive a contrast media as part of a medical imaging procedure. The database 310 may be stored in a memory associated with a computer device wherein the computer device may be in communication with the computer network 300 as shown in FIG. 3. Thus, the database 310 may be interrogated by, for instance, the imaging device 110, dispensing device 120, and/or analyzing device 130 such that an operator of the medical imaging system and dispensing system of the present invention may gain access to the data stored in the database 310. Thus, in some cases, wherein for instance, a patient must undergo multiple medical imaging procedures, the dispensing device 120 may interrogate the database 310 to determine the level of the at least one substance in a biological fluid sample taken from the patient prior to a first medical imaging procedure. In addition, the database may be interrogated by medical professionals seeking patient history related to, for instance, the patient's renal function, and/or history of medical imaging procedures.

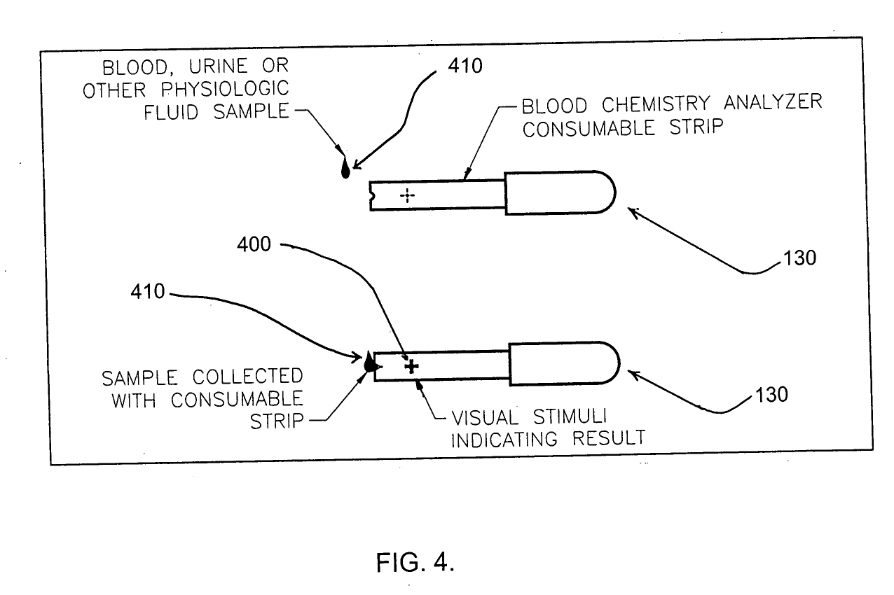

According to other embodiments, as shown in FIG. 4, the analyzing device 130 of the medical imaging system of the present invention may alternatively comprise a self-contained consumable testing device 130 configured to be in fluid communication with a biological fluid sample 410. Furthermore, the testing device 130 may be further configured to provide a visual indicia 400 to advise the operator of the system of the level of the at least one substance relative to the selected range. The self-contained consumable testing device 130 may further comprise a capillary configured to receive the biological fluid sample 410 from a prospective patient. The self-contained consumable testing device may further comprise a reagent adapted to react with at least one substance in the biological fluid sample 410 such that the reagent may produce a visual indicia 400, such as for instance, a color change, and/or a symbolic indicia to indicate that the level of at least one substance is within a selected range such that a contrast media may be safely dispensed to the patient in conjunction with a medical imaging procedure. According to this embodiment of the medical imaging system of the present invention, a plurality of self-contained consumable testing devices 130 may be made available in the medical imaging suite 100 such that an operator of the medical imaging system may quickly determine, via the self-contained consumable testing device 130, if a particular patient may be eligible to safely receive an administration of a contrast media used in a medical imaging procedure. The embodiment of the medical imaging system and/or dispensing system of the present invention comprising a self-contained consumable testing device 130 (as shown in FIG. 4) may be preferable for use in hospitals wherein existing medical imaging suites exist having medical imaging devices 110 and/or dispensing devices 120 that are non-network capable, or where cost restrictions prevent the purchase of a computer device-based analyzing device 130.

The present invention also provides method embodiments for assessing the renal function of a patient prior to the dispensing of a contrast media as part of a medical imaging procedure such that the assessment may occur without sending the prospective patient and/or a biological fluid sample associated with the prospective patient outside the medical imaging suite 100. According to one embodiment, the method comprises the steps of: collecting a biological fluid sample from the patient; determining a level of at least one substance in the biological fluid sample of the patient using an analyzing device 130 located in a medical imaging suite 100; comparing the level of the at least one substance to a selected range of levels of the at least one substance using the analyzing device 130 located in the medical imaging suite 100; and advising an operator of the analyzing device as to whether the level is within the selected range such that the operator may be advised of the patient's renal function prior to dispensing a contrast media as part of a medical imaging procedure.

According to other method embodiments, the method may further comprise the step of dispensing the contrast media to the patient using a dispensing device 120 if the level of the at least one substance is within the selected range. As such, this embodiment of the method may pre-screen the patient for a substantially normal renal function prior to dispensing the contrast media via the dispensing device 120. According to other method embodiments, the determining step may further comprise determining a level of blood urea nitrogen (BUN), creatinine, or combinations thereof in the biological fluid sample.

The present invention also provides computer program product embodiments capable of executing the various method steps of the present invention. According to some embodiments, the computer program product may be executable on the computer device 220, dispensing device 120, and/or imaging device 110. The computer program embodiments of the present invention may be further configured to receive patient physiological data including, but not limited to parameters such as height, weight, sex, age, pre-existing medical conditions, patient-identifier information, and other data that may be relevant to the medical imaging procedure. Such data may also, in some embodiments, include other information such as time, date, location of medical imaging procedure, lot numbers for various pharmaceuticals, contrast media, or other medical supplies used in the medical imaging procedure, and/or other data related to the medical imaging procedure.

According to some embodiments, the data described above may be received by the computer device 220, dispensing device 120, and/or imaging device 110 via the computer program product embodiments from the database 310 or from a user interface, such as a keyboard, mouse, touch screen or other user interface that may be operably engaged with and/or in communication with (via wire or wireless methods) the computer device 220, dispensing device 120, and/or imaging device 110. The computer program product embodiments may also be configured to receive the level of the at least one substance (such as, for instance, blood urea nitrogen (BUN), creatinine, or combinations thereof) in the biological fluid sample that may be determined by the analyzing device 130 and determine, for instance, based on the received data, if an alternate volume, type, concentration, and/or combination of one or more contrast media may be properly and safely administered to the patient by the dispensing device 120 such that the contrast media may be safely cleared by the renal function of the patient.

Many modifications and other embodiments of the invention will come to mind to one skilled in the art to which this invention pertains having the benefit of the teachings presented in the foregoing descriptions and the associated drawings. For example, one skilled in the art will appreciate that the systems, methods, and computer program products disclosed herein may also be used to determine a level of at least one substance in the biological fluid sample so as to enable the further determination of a corresponding volume, type, concentration, and/or combination of one or more contrast media that may be properly and safely administered to a patient such that the contrast media may be safely cleared by the renal function of the patient. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A medical imaging system comprising:
a medical imaging device configured to provide an image of a patient using a contrast media dispensed to the patient;
a dispensing device configured to dispense the contrast media to the patient;
an analyzing device adapted to receive and analyze a biological fluid sample from the patient so as to determine a level of at least one substance in the biological fluid sample, the analyzing device being adapted to advise an operator of the system of the level of the at least one substance, the analyzing device being further adapted to advise the operator if the level of the at least on substance is within a selected range.

2. A medical imaging system according to claim 1, wherein the analyzing device is further adapted to advise the operator to dispense the contrast media if the level of the at least one substance is within the selected range.

3. A medical imaging system according to claim 1, wherein the analyzing device is further configured to be in communication with the dispensing device so as to send the level of the at least one substance thereto, and wherein the dispensing device is further configured to receive the level of the at least one substance and to dispense the contrast media to the patient if the level of the at least one substance is within the selected range.

4. A medical imaging system according to claim 1, wherein the at least one substance is selected from the group consisting of:
blood urea nitrogen (BUN);
creatinine; or
combinations thereof.

5. A medical imaging system according to claim 1, wherein the medical imaging device, the dispensing device, and the analyzing device are co-located in a medical imaging suite so as to determine the level of the at least one substance in the medical imaging suite prior to a medical imaging procedure.

6. A medical imaging system according to claim 1, wherein the analyzing device further comprises a testing device configured to be in fluid communication with the biological fluid sample such that the testing device is further configured to provide a visual indicia to advise the operator of the system of the level of the at least one substance relative to the selected range.

7. A medical imaging system according to claim 1, wherein the analyzing device further comprises:
a testing device configured to receive the biological fluid sample and to be in fluid communication therewith; and
a computer device configured to receive the testing device and to become operably engaged therewith to determine the level of the at least one substance in the biological fluid sample.

8. A medical imaging system according to claim 7, wherein the testing device further comprises a biological fluid sample collection reservoir, at least one reagent configured to interact with the at least one substance and a connecting device configured to communicate with the computer device, and wherein the computer device is further configured to communicate with the testing device so as to determine the level of the at least one substance.

9. A medical imaging system according to claim 8, wherein the computer device further comprises a display configured to provide visual indicia so as to advise the operator of the system of the level of the at least one substance.

10. A medical imaging system according to claim 8, wherein the computer device further comprises an input device configured to receive data selected from the group consisting of:
data related to the patient;
data related to the selected range; and
combinations thereof.

11. A dispensing system adapted to dispense a contrast media used in a medical imaging procedure, the dispensing system comprising:
a dispensing device configured to dispense the contrast media to a patient;
an analyzing device adapted to receive and analyze a biological fluid sample from the patient so as to determine a level of at least one substance in the biological fluid sample, the analyzing device being adapted to advise an operator of the system of the level of the at least one substance, the analyzing device being further adapted to advise the operator if the level of the at least on substance is within a selected range.

12. A dispensing system according to claim 11, wherein the analyzing device is further adapted to advise the operator to dispense the contrast media if the level of the at least one substance is within the selected range.

13. A dispensing system according to claim 11, wherein the analyzing device is further configured to cooperate with the dispensing device such that the dispensing device is further configured to receive the level of the at least one substance and to dispense the contrast media to the patient if the level of the at least one substance is within the selected range.

14. A dispensing system according to claim 11, wherein the at least one substance is selected from the group consisting of:
blood urea nitrogen (BUN);
creatinine; or
combinations thereof.

15. A dispensing system according to claim 11, wherein the dispensing device and the analyzing device are co-located in a medical imaging suite so as to determine the level of the at least one substance in the medical imaging suite prior to dispensing the contrast media for a medical imaging procedure.

16. A dispensing system according to claim 11, wherein the analyzing device further comprises a testing device configured to be in fluid communication with the biological fluid sample such that the testing device is further configured to provide a visual indicia to advise the operator of the system of the level of the at least one substance relative to the selected range.

17. A dispensing system according to claim 11, wherein the analyzing device further comprises:
a testing device configured to receive the biological fluid sample and to be in fluid communication therewith; and
a computer device configured to receive the testing device and to become operably engaged therewith to determine the level of the at least one substance in the biological fluid sample.

18. A dispensing system according to claim 17, wherein the testing device further comprises a biological fluid sample reservoir, at least one reagent configured to interact with the at least one substance and a transmitting device configured to communicate with the computer device, and wherein the computer device is further configured to communicate with the testing device so as to determine the level of the at least one substance.

19. A dispensing system according to claim 18, wherein the computer device further comprises a display configured to provide visual indicia so as to advise the operator of the system of the level of the at least one substance.

20. A dispensing system according to claim 18, wherein the computer device further comprises an input device configured to receive data selected from the group consisting of:
data related to the patient;
data related to the selected range; and
combinations thereof.

21. A method for assessing renal function of a patient prior to the dispensing of a contrast media as part of a medical imaging procedure, the method comprising:
collecting a biological fluid sample from the patient;
determining a level of at least one substance in the biological fluid sample of the patient using an analyzing device located in a medical imaging suite;
comparing the level of the at least one substance to a selected range of levels of the at least one substance using the analyzing device located in the medical imaging suite; and
advising an operator of the analyzing device as to whether the level is within the selected range.

22. A method according to claim 21, further comprising dispensing the contrast media to the patient if the level of the at least one substance is within the selected range such that the patient is screened for substantially normal renal function prior to dispensing the contrast media.

23. A method according to claim 21, wherein the determining step further comprises determining a level of a substance in the biological fluid sample, the substance selected from the group consisting of:
blood urea nitrogen (BUN);
creatinine; or
combinations thereof.

24. A method according to claim 21 further comprising adjusting a property of the contrast media to create an altered contrast media if the level of the at least one substance is outside the selected range such that the altered contrast media may be cleared by renal function of the patient after dispensing the altered contrast media.

25. A method according to claim 24 wherein the property of the contrast media is selected from the group consisting of:
volume of the contrast media;
delivery rate of the contrast media;
concentration of the contrast media;
type of the contrast media; or
combinations thereof.

26. A computer program product capable of controlling an analyzing device and a dispensing device located in a medical imaging suite for assessing renal function of a patient prior to the dispensing of a contrast media as part of a medical imaging procedure, the computer program product comprising a computer-readable storage medium having computer-readable program code portions stored therein, the computer-readable program code portions comprising:
an executable portion for determining a level of at least one substance in a biological fluid sample taken from the patient using the analyzing device;
an executable portion for comparing the level of the at least one substance to a selected range of levels of the at least one substance using the analyzing device; and
an executable portion for advising an operator of the analyzing device as to whether the level is within the selected range.

27. A computer program product according to claim 26, further comprising an executable portion for dispensing the contrast media to the patient using the dispensing device if the level of the at least one substance is within the selected range such that the patient is screened for substantially normal renal function prior to dispensing the contrast media.

28. A computer program product according to claim 26, further comprising an executable portion for adjusting a property of the contrast media using the dispensing device to create an altered contrast media if the level of the at least one substance is outside the selected range such that the altered contrast media may be cleared by the renal function of the patient after dispensing the altered contrast media.

29. A computer program product according to claim 28 wherein the property of the contrast media is selected from the group consisting of:
volume of the contrast media;
delivery rate of the contrast media;
concentration of the contrast media;
type of the contrast media; or
combinations thereof.
